(19) United States
(12) Patent Application Publication
(10) Pub. No.: US 2011/0305651 A1
(43) Pub. Date: Dec. 15, 2011

(54) SUNLESS TANNING COMPOSITIONS WITH NUCLEOPHILE FUNCTIONALIZED SULFONIC ACIDS

(75) Inventors: Joseph Oreste Carnali, Trumbull, CT (US); Wei-min Peng, Shanghai (CN); Qiang Qiu, Shanghai (CN); Zhijian Yang, Shanghai (CN); Xiaoxia Yang, Shanghai (CN)

(73) Assignee: CONOPCO, INC. D/B/A UNILEVER, Englewood Cliffs, NJ (US)

(21) Appl. No.: 13/155,451

(22) Filed: Jun. 8, 2011

(30) Foreign Application Priority Data
Jun. 15, 2010 (CN) PCT/CN2010/000864

(51) Int. Cl. A61K 8/46; A61Q 17/04

(52) U.S. Cl. 424/59

(57) ABSTRACT

Sunless tanning compositions with nucleophile functionalized sulfonic acids are described. The compositions are stable and result in excellent tanning results when sunless tanning agents like 1,3-dihydroxy-2-propanone are used.

### FIELD OF THE INVENTION

The present invention is directed to a composition and method for imparting a sunless tan to skin. More particularly, the invention is directed to a composition and method that employ a sunless tanning agent as well as an adjuvant for the sunless tanning agent. The composition employs a nucleophile functionalized sulfonic acid as an adjuvant, is stable, and when applied, unexpectedly results in the consumer having skin with a brownish/tan coloration within a consumer acceptable time.

### BACKGROUND OF THE INVENTION

Sunless tanning agents are typically formulated into two types of cosmetic products. Of these, the most traditional is the self-tanning lotion. The imparted benefit is to achieve a skin coloration equivalent to that from basking in the sun. More recently, a second product category has arrived. Therein a sunless tanning agent in small amounts is added to a typical moisturizing lotion. A “glow or shine” is thereby imparted. Glow or shine is a major factor in the appearance of healthy looking skin.

Prominent among the sunless tanning agents is dihydroxyacetone (“DHA” which is also chemically known as 1,3-dihydroxy-2-propanone). DHA, after application, is believed to exert its effect through interactions between its carbonyl group and the amino groups of amino acids and peptides naturally occurring in the hydrolipid pellicle and first layers of the stratum corneum of the skin. These so-called Maillard reactions are believed (see, e.g., Bobin et al., J. Soc. Cosmet. Chem. 35: 255 (1984)) to lead to formation of brown pigments in the skin, thereby giving skin an appearance similar to that of a naturally obtained tan. A number of other sunless tanning agents have been identified, including melanin, mahakanni (eclipta alba), methyl glyoxal, erythrulose, alloxan, 2,3-dihydroxysuccindialdehyde, mixtures thereof, or the like. These agents are believed to work in a similar way as DHA, but typically do yield somewhat of their own unique pigmentation.

Unfortunately, many sunless tanning products available on the market are not stable in that they turn a yellow and/or orange color after application, especially when exposed to UV light. Other sunless tanning products perform poorly and do not quickly impart a noticeable brown color after application. Such poorly performing products do not prevent “tan-happy” consumers from basking in the sun. Products that underperform, therefore, do not protect consumers from the sun's ultraviolet rays.

There is increasing interest to develop compositions and methods for imparting a sunless tan, and especially, compositions that are storage stable. This invention, therefore, is directed to a composition and method that employ a sunless tanning agent as well as an adjuvant for the sunless tanning agent. The composition employs a nucleophile functionalized sulfonic acid as an adjuvant and, when applied, unexpectedly results in the consumer having skin with a brownish/tan coloration within a consumer acceptable time.

### Additional Information

Efforts have been disclosed for making self-tanning cosmetic compositions. In U.S. Pat. Nos. 5,232,688 and 5,612,044, self-tanner compositions with DHA are described.

Other efforts have been disclosed for making self-tanning compositions. In U.S. Pat. No. 5,750,092, compositions with DHA and secondary amines are described.

Still other efforts have been disclosed for making self-tanning compositions. In U.S. Pat. No. 6,231,837, self tanning formulations comprising DHA, polyethoxyglycol and a polyol are described. In U.S. Pat. No. 5,756,075, a self tanning method is disclosed in which a fluid comprising DHA can be mixed with a complimentary fluid comprising specific primary amines.

None of the additional information describes a method and/or composition that yield excellent sunless tanning results whereby the composition and method employ a sunless tanning agent and a nucleophile functionalized sulfonic acid as an adjuvant.

### SUMMARY OF THE INVENTION

In a first aspect, the present invention is directed to a composition comprising:
a) a sunless tanning agent; and
b) an adjuvant for the sunless tanning agent, the adjuvant comprising a nucleophile functionalized sulfonic acid.

In a second aspect, the present invention is directed to a method for generating a sunless tan comprising the step of applying to the skin the composition of the first aspect of this invention.

All other aspects of the present invention will more readily become apparent upon considering the detailed description and examples which follow.

Sunless tanning, as used herein, means obtaining the suntan look by applying a topical composition. The same can be interchanged with self-tanning. Composition, as used herein, is meant to include a substance applied to a human body for imparting a sunless tan where the composition is for example, an end use leave-on skin lotion, cream or mouse, shampoo, hair conditioner, shower gel, toilet bar, body wash, shaving cream, body wax, depilatory, mascara, sunscreen product or the like. Such a composition may also be put on body towelettes for application to the body. In a preferred embodiment, the composition of this invention is a lotion or cream. Consumer acceptable time means within about 3 to about 6 hours from application, and preferably, from about 1 to about 2 hours, and most preferably, from about 15 to about 30 minutes subsequent to application. Stable means at least about 60%, and preferably, at least about 85% by weight of the sunless tanning agent in the composition does not result in formation of color bodies after about twenty (20) days of storage at about 35° C. and when formulated as a monophase product with adjuvant and at a pH in a range from 3 to 6. Nucleophile functionalized sulfonic acid means having an —SO₃H group with a nucleophilic group attached directly thereon or attached thereon via a carbon comprising bridging radical whereby the same is meant to include any salt derivatives thereof. Nucleophile, as used herein, means a component able to donate electrons. Carbon comprising bridging radical includes an alkyl or alkenyl bridging radical which may be divalent, substituted, unsubstituted, linear, branched or cyclic where cyclic includes aryl groups.

Comprising, as used herein meant to include consisting essentially of and consisting of. All ranges identified herein are meant to include all ranges subsumed therein if, for example, reference to the same is not explicitly made.

### DETAILED DESCRIPTION OF THE INVENTION

The sunless tanning agent suitable for use in this invention is only limited to the extent that the same may be applied topically on humans to form pigmented components. Such materials may be alpha-hydroxyaldelydes and ketones, glyceraldehyde, troxerutin and related alcohol aldehydes, various indoles, imidazoles and derivatives thereof.

Illustrative yet non-limiting examples of the sunless tanning agents that may be used in this invention include dihydroxyacetone, melanin, mahakanni (eclipta alba), methyl glyoxal, erythrulose, alloxan, 2,3-dihydroxysuccindialdehyde, mixtures thereof, or the like. In a preferred embodiment, the sunless tanning agent used is dihydroxyacetone, erythrulose, alloxan, or a mixture thereof. In a most preferred embodiment, the sunless tanning agent is dihydroxyacetone.

Typically, the sunless tanning agent makes up from about 0.025 to about 35%, and preferably, from about 0.05 to about 15%, and most preferably, from about 0.5 to about 10% by weight of the composition, based on total eight of the composition and including all ranges subsumed therein.

The adjuvant (i.e., nucleophile functionalized sulfonic acid) that may be used in this invention is limited only to the extent that the same may be used in a composition suitable for topical application to humans.

Typically, the adjuvant is represented by the formula:

Nu-X—SO₃H I

wherein X is a C₁₋₁₀ alkyl or alkenyl bridging radical;

Nu is —SR, —CN, I, —OR, Cl or —NHR; and

each R is independently H or a C₁₋₁₀ substituted or unsubstituted alkyl.

In a preferred embodiment, the adjuvant used in this invention is represented as:

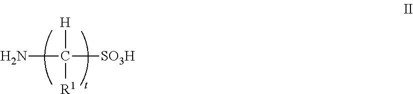

where R¹ is H or a C₁₋₁₀ substituted or unsubstituted alkyl group, and t is an integer from about 1 to about 8.

In an especially preferred embodiment, the sunless tanning adjuvant used in this invention is an amino n-alkyl sulfonic acid, and most desirably, aminomethanesulfonic acid, 2-aminoethanesulfonic acid (taurine), 3-propane-sulfonic acid (homotaurine), or mixtures thereof. Typically, the adjuvant makes up from about 0.025 to about 35%, and preferably, from about 0.05 to about 15%, and most preferably, from about 0.5 to about 8% by weight of the composition, including all ranges subsumed therein. It is also within the scope of this invention to employ mixtures of any of the adjuvants represented by the above-identified formulae I and II.

Compositions of the present invention will typically include a cosmetically acceptable carrier. Water is the most preferred carrier. Amounts of water may range from about 1 to about 99%, and preferably, from about 5 to about 90%, and most preferably, from about 35 to about 80% and optimally from about 40 to about 75% by weight, based on total weight of the composition and including all ranges subsumed therein. Ordinarily the compositions will be water and oil emulsions, most preferably, of the oil-in-water variety. Water-in-oil emulsions, and especially, those generally classified as water-in-oil and high internal phase emulsions are, however, an option. Illustrative examples of the high internal phase emulsions suitable to carry the actives and adjuvants of this invention described in commonly owned U.S. Patent Application Publication Nos. 2008/0311058 and 2009/0217415, the disclosures of which are incorporated herein by reference.

Other cosmetically acceptable carriers may include mineral oils, silicone oils, synthetic or natural esters, fatty acids and alcohols. Amounts of these materials may range from about 0.1 to about 50%, and preferably, from about 0.1 to about 30%, and most preferably, from about 1 to about 20% by weight of the composition, including all ranges subsumed therein.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from about 3 to about 9, and preferably, from about 4 to about 5 silicon atoms.

Linear volatile silicone materials generally have viscosities of less than about 5 centistokes at 25° C. while cyclic materials typically have viscosities of less than about 10 centistokes.

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone) with viscosities of from about 5 to about 100,000 centistokes at 25° C.

An often preferred silicone source is a cyclopentasiloxane and dimethiconol solution.

Among suitable esters are:
(1) Alkenyl or alkyl esters of fatty acids having 10 to 20 carbon atoms like isopropyl palmitate, isopropyl isostearate, isononyl isonanonoate, oleyl myristate, oleyl stearate, and oleyl oleate;
(2) Ether-esters such as fatty acid esters of ethoxylated fatty alcohols;
(3) Polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxy-ethylene sorbitan fatty acid esters;
(4) Wax esters such as beeswax, spermaceti, myristyl myristate, stearyl stearate; and
(5) Sterol esters, of which soya sterol and cholesterol fatty acid esters are examples thereof.

Fatty acids having from 10 to 30 carbon atoms may be included in the compositions of this invention. Illustrative of this category are pelargonic, lauric, myristic, palmitic, stearic, isostearic, hydroxystearic, oleic, linoleic, ricinoleic, arachidic, behenic and erucic acids.

Emulsifiers may be present in the compositions of the present invention. Total concentration of the emulsifier may range from about 0.1 to about 40%, and preferably, from about 1 to about 20%, and most preferably, from about 1 to about 5% by weight of the composition, including all ranges subsumed therein. The emulsifier may be selected from the group consisting of anionic, nonionic, cationic and amphoteric actives. Particularly preferred nonionic actives are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Preferred anionic emulsifiers include soap, alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, alkylethercarboxylates and combinations thereof.

Cationic emulsifiers that may be used include, for example, palmitamidopropyltrimonium chloride, distearyldimonium chloride and mixtures thereof. Useful amphoteric emulsifiers include cocoamidopropyl betaine, C₁₂-C₂₀ trialkyl betaines, sodium lauroamphoacetate, and sodium laurodiamphoacetate or a mixture thereof.

Other generally preferred emulsifiers include glyceryl stearate, glycol stearate, stearamide AMP, PEG-100 stearate, cetyl alcohol as well as emulsifying/thickening additives like hydroxyethylacrylate/sodium acryloyldimethyl taurates copolymer/squalane and mixtures thereof.

Preservatives can desirably be incorporated into the compositions comprising the sunless tanning agent and adjuvant of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are iodopropynyl butyl carbamate, phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients in the emulsion. Preservatives are preferably employed in amounts ranging from about 0.01% to about 2% by weight of the composition, including all ranges subsumed therein.

Thickening agents may be included in compositions of the present invention. Particularly useful are the polysaccharides. Examples include starches, natural/synthetic gums and cellulosics. Representative of the starches are chemically modified starches such as sodium hydroxypropyl starch phosphate and aluminum starch octenylsuccinate. Tapioca starch is often preferred. Suitable gums include xanthan, sclerotium, pectin, karaya, arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloyldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel INS100.

Amounts of the thickener may range from about 0.001 to about 5%, and preferably, from about 0.1 to about 2%, and most preferably, from about 0.2 to about 0.5% by weight of the composition including all ranges subsumed therein.

Fragrances, fixatives and abrasives may optionally be included in compositions of the present invention. Each of these substances may range from about 0.05 to about 5%, preferably between 0.1 and 3% by weight.

To enhance skin moisturization, cationic ammonium compounds may optionally be used in the compositions of this invention. Such compounds include salts of hydroxypropyltri (C₁-C₃ alkyl) ammonium mono-substituted-saccharide, salts of hydroxypropyltri (C₁-C₃ alkyl) ammonium mono-substituted polyols, dihydroxypropyltri (C₁-C₃ alkyl) ammonium salts, dihydroxypropyldi (C₁-C₃ alkyl) mono(hydroxyethyl) ammonium salts, guar hydroxypropyl trimonium salts, 2,3-dihydroxypropyl tri(C₁-C₃ alkyl or hydroxalkyl) ammonium salts or mixtures thereof. In a most preferred embodiment and when desired, the cationic ammonium compound employed in this invention is the quaternary ammonium compound 1,2-dihydroxypropyltrimonium chloride. If used, such compounds typically make up from about 0.01 to about 30%, and preferably, from about 0.1 to about 15% by weight of the composition.

When cationic ammonium compounds are used, preferred additives for use with the same are moisturizing agents such as substituted ureas like hydroxymethyl urea, hydroxyethyl urea, hydroxypropyl urea; bis(hydroxymethyl)urea; bis(hydroxyethyl)urea; bis(hydroxypropyl)urea; N,N′-dihydroxymethyl urea; N,N′-di-hydroxyethyl urea; N,N′-di-hydroxypropyl urea; N,N,N′-tri-hydroxyethyl urea; tetra(hydroxymethyl)urea; tetra(hydroxyethyl)urea; tetra(hydroxypropyl)urea; N-methyl-N′-hydroxyethyl urea; N-ethyl-N,N—N′-hydroxyethyl urea; N-hydroxypropyl-N′-hydroxyethyl urea and N,N′-dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-1-propyl or 2-hydroxy-1-propyl radicals. Most preferred is hydroxyethyl urea. The latter is available as a 50% aqueous liquid from the National Starch & Chemical Division of ICI under the trademark Hydrovance.

Amounts of substituted urea, when used, in the composition of this invention range from about 0.01 to about 20%, and preferably, from about 0.5 to about 15%, and most preferably, from about 2 to about 10% based on total weight of the composition and including all ranges subsumed therein.

Conventional humectants may be employed in the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerin, propylene glycol or a mixture thereof. The amount of humectant employed may range anywhere from 0.5 to 20%, preferably between 1 and 15% by weight of the composition.

When cationic ammonium compound and substituted urea are used, in a most especially preferred embodiment at least from about 1 to about 15% glycerin is used, based on total weight of the composition and including all ranges subsumed therein.

Compositions of the present invention may include vitamins. Illustrative vitamins are Vitamin A (retinol), Vitamin B₂, Vitamin B₃ (niacinamide), Vitamin B₆, Vitamin C, Vitamin E, Folic Add and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present in compositions according to the present invention may range from 0.001 to 10%, preferably from 0.01% to 1 optimally from 0.1 to 0.5% by weight of the composition.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.01 to about 15% by weight of the composition.

A variety of herbal extracts may optionally be included in compositions of this invention. The extracts may either be water soluble or water-insoluble carded in a solvent which respectively is hydrophilic or hydrophobic. Water and ethanol are the preferred extract solvents. Illustrative extracts include those from green tea, yarrow, chamomile, licorice, aloe vera, grape seed, citrus unshui, willow bark, sage, thyme and rosemary.

Also optionally suitable for use include materials like sunscreens as well as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials may range from about 0.000001 to about 10%, preferably from about 0.0001 to about 1% by weight of the composition.

Conventional buffers/pH modifiers may be used. These include commonly employed additives like sodium hydroxide, hydrochloric acid, citric acid and citrate/citric acid buffers. In an especially preferred embodiment, the pH of the composition of this invention is from about 3 to about 6, and preferably, from about 3.25 to about 4.75, and most preferably, from about 3.25 to about 4, including all ranges subsumed therein.

Colorants, ° pacifiers, chelators (like tetrasodium EDTA) and abrasives may also be included in the compositions of the present invention. Each of these substances may range from about 0.05 to about 5%, preferably between 0.1 and 3% by weight of the composition.

In an especially preferred embodiment, the composition of the present invention comprises less than about 5%, and preferably, from 0.01 to 4% by weight glycine, and most preferably, no glycine.

A wide variety of packaging can be employed to store and deliver the compositions. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of personal care products may be delivered in a stick composition formulation in a container with propel-repel mechanism where the stick moves on a platform towards a dispensing orifice. Metallic cans pressurized by a propellant and having a spray nozzle serve as packaging for antiperspirants, shave creams and other personal care products. Toilette bars may have packaging constituted by a cellulosic or plastic wrapper or within a cardboard box or even encompassed by a shrink wrap plastic film.

Optionally, the composition of this invention may be divided so that a first portion may carry sunless tanning agent and a second portion may carry adjuvant. When dividing the composition, each portion should be packaged separately from each other and not come into contact with each other until application to the body. The packaging for dual compositions is known and commercially available. Upon application, the make up of the composition the combined portions) is as described herein.

When making the composition of the present invention, ingredients may be combined in no particular order. Typically the ingredients are combined and mixed under conditions of moderate shear and at ambient temperature with pressure being atmospheric conditions. In a most preferred embodiment, DHA and adjuvant are not added at a time when mixing and heating are desired. When applied by the consumer, typically from about 1 to 5 mg, and preferably, from about 1 to 4 mg, and preferably, from about 1.5 to 2.5 mg of composition is applied to about 1 centimeter squared of body surface (like skin) and including all ranges subsumed therein.

The following examples are provided to facilitate an understanding of the present invention. The examples are not intended to limit the scope of the claims.

### Example 1

Compositions with 2.5% DHA were made and adjusted to pH 5.5 with a 0.2 M citrate/citric acid buffer solution. Composition without adjuvant constitutes comparative sample C1. This pH was chosen to mimic pH conditions at the surface of skin. Comparative sample C2 was made to contain 1% glycine in addition to DHA. Inventive examples E1-E3 were prepared with adjuvants of this invention and free of glycine. Aminomethane sulfonic acid (AMSA), aminoethane sulfonic acid (touring, and aminopropane sulfonic acid (homotaurine) were used, respectively, at levels molar equivalent to 1% glycine as shown in the Table below. All ingredients were added on a percent by weight basis and mixed with moderate shear under atmospheric conditions.

TABLE

| Sample | | | | | |
|---|---|---|---|---|---|
| Ingredient* | C1 | C2 | E1 | E2 | E3 |
| DHA | 2.5 | 2.5 | 2.5 | 2.5 | 2.5 |
| Glycine | 0 | 1.0 | 0 | 0 | 0 |
| AMSA | 0 | 0 | 1.48 | 0 | 0 |
| Taurine | 0 | 0 | 0 | 1.62 | 0 |
| Homotaurine | 0 | 0 | 0 | 0 | 1.86 |
| 0.2M citrate/ | Balance | Balance | Balance | Balance | balance |
| citric acid buffer | | | | | |
| pH 5.5 | | | | | |

*all adjuvants were supplied by Sigma-Aldrich Co.

The action of adjuvants used was demonstrated on synthetic skin made commercially available as VITRO-SKIN® from IMS Inc. The substrate (about 600 cm²) was pre-hydrated (about 100 cm² at a time) before use by storing the same for 24 hours in a desiccator containing a 15% glycerol-in-water solution (95% RH). The substrate was cut into squares (about 6 cm²) and treated with 25 μL of each sample, spread smoothly over a circular area of about 5 cm². The squares of treated substrate were then stored at 35° C. and 40% RH for up to about 48 hours, during which time any color development was monitored periodically using a HunterLab Labscan XE colorimeter. The overall color of the treated substrates was reported in terms of the Individual Typologic Angle ITA°=180 (Arc Tangent (L*−50/b*)/π, as defined by A. Chardon et al. in the International Journal of Cosmetic Science, volume 13, pages 191-208 (1991), where Arc Tangent is expressed in radians, ITA° is recognized as a quantitative and objective means of classifying individual skin color and skin melanization levels. The higher the ITA°, the lighter the skin.

After a period of 24 and 48 hours, the change in ITA°, relative to that of untreated synthetic skin and labeled Δ ITA°, is shown in Table I below. A larger Δ ITA° signifies darker tanning or coloring.

TABLE I

| | Δ ITA° | Δ ITA° |
|---|---|---|
| Sample | after 24 hrs. | After 48 hrs. |
| C1 | 10 ± 2 | 22 ± 2 |
| C2 | 35 ± 2 | 42 ± 2 |
| E1 | 30 ± 2 | 38 ± 2 |
| E2 | 38 ± 2 | 45 ± 2 |
| E3 | 25 ± 2 | 32 ± 2 |

Compared to control depete of adjuvant, compositions made according to this invention and having nucleophile functionalized sulfonic acid adjuvants (i.e., E1-E3) displayed a darkness of tan, as gauged by Δ ITA°, at an amount similar to or better than that of DHA and glycine (C2) and significantly better than DHA used without adjuvant (C1). Unexpectedly, the adjuvant action of the nucleophile functionalized sulfonic acids of this invention resulted in at least commercially acceptable tanning results when DHA and no glycine was used in the composition.

### Example 2

Chemical storage stability of DHA in the presence of the adjuvants of this invention was assessed by storing the following comparative and inventive samples described in Table II for up to 300 hours at a temperature of about 50° C. The pH of the samples (established with the buffer described in Example 1) was set at 15 to simulate a typical pH for end use compositions. During storage, the turbidity of the each sample was monitored periodically using a Carey Spectrophotometer.

TABLE II

| Sample | | | | | |
|---|---|---|---|---|---|
| Ingredient | C3 | C4 | E4 | E5 | E6 |
| DHA | 2.5 | 2.5 | 2.5 | 2.5 | 2.5 |
| Glycine | 0 | 1.0 | 0 | 0 | 0 |
| AMSA | 0 | 0 | 1.48 | 0 | 0 |
| Taurine | 0 | 0 | 0 | 1.62 | 0 |
| Homotaurine | 0 | 0 | 0 | 0 | 1.86 |
| 0.2M citrate/ | Balance | Balance | Balance | Balance | Balance |
| citric acid | | | | | |
| buffer pH 3.5 | | | | | |

Storage stability of DHA in the above samples, as gauged by optical density (at 600 nm) developed upon storage, is reported as slope of resulting absorbance versus time plot in Table III.

TABLE III

| Sample | Delta A @ 600 nm/delta (hrs.) |
|---|---|
| C3 | 0.000 |
| C4 | 0.007 |
| E4 | 0.009 |
| E5 | 0.004 |
| E6 | 0.004 |

DHA was found to be stable at pH 3.5 for the 300 hour duration of the test (C3). The presence of glycine (C4) gave rise to a noticeable discoloration. For the series of nucleophile functionalized sulfonic acids, the discoloration for AMSA (E1) was similar to that of glycine, but taurine (E5) and homotaurine (E6) resulted in very stable systems, performing about equally well to each other. The results unexpectedly reveal that the stability of compositions made only with the adjuvants of this invention and DHA were about as stable as or more stable than compositions made with DHA and glycine (the conventionally used adjuvant).

### Example 3

The utility of the inventive adjuvants in sunless-tanning compositions is illustrated in the following samples shown in Table IV, all of which were made by mixing ingredients with moderate shear under atmospheric conditions.

TABLE IV

| | Sample (% by weight) | | |
|---|---|---|---|
| Ingredient | E7 | E8 | E9 |
| Sodium hydroxypropyl | 0.10 | 0.10 | 0.10 |
| starch phosphate | | | |
| Chelator | 0.11 | 0.11 | 0.11 |
| Preservative | 0.3 | 0.3 | 0.3 |
| Glycerin | 12.0 | 12.0 | 12.0 |
| Citric acid* | 0.1-0.2 | 0.1-0.2 | 0.1-0.2 |
| Colorant | 0.19 | 0.19 | 0.19 |
| Stearic acid | 1.97 | 1.97 | 1.97 |
| Emulsifier | 4.2 | 4.2 | 4.2 |
| Cetyl alcohol | 0.31 | 0.31 | 0.31 |
| Isopropyl palmitate | 2.25 | 2.25 | 2.25 |
| Silicone oil | 1.5 | 1.5 | 1.5 |
| Phenoxyethanol | 0.4 | 0.4 | 0.4 |
| Perfume | 0.35 | 0.35 | 0.35 |
| Dihydroxyacetone | 2.5 | 0.0 | 0.0 |
| Alloxane | 0.0 | 2.5 | 0.0 |
| Erythrulose | 0.0 | 0.0 | 2.5 |
| Taurine | 1.0 | 1.0 | 1.0 |
| Deionized water | balance | balance | balance |

*to pH of about 3.5

Samples E7 to E9 were assessed for efficacy and all gave coloration to synthetic skin, demonstrating that the inventive adjuvants work well across a broad range of sunless tanning actives and in compositions ready for use by consumers.

### Example 4

The utility of the inventive ingredients in sunless-tanning compositions was also illustrated in the following samples shown in Table V, all of which were prepared as described above.

TABLE V

| | Sample (% by weight) | | |
|---|---|---|---|
| Ingredients | E10 | E11 | E12 |
| Thickening agent | 0.25 | 0.25 | 0.25 |
| Tapioca starch | 0.5 | 0.5 | 0.5 |
| Chelator | 0.05 | 0.05 | 0.05 |
| Preservative | 0.3 | 0.3 | 0.3 |
| Glycerin | 12.0 | 12.0 | 12.0 |
| Citric acid* | 0.02 | 0.02 | 0.02 |
| Colorant | 0.19 | 0.19 | 0.19 |
| Glyceryl stearate | 2.4 | 2.4 | 2.4 |
| PEG 100 stearate | 1.2 | 1.2 | 1.2 |
| Cetyl alcohol | 2.4 | 2.4 | 2.4 |
| Isopropyl palmitate | 2.0 | 2.0 | 2.0 |
| Wax | 1.0 | 1.0 | 1.0 |
| Dimethicone 50 cst | 3.0 | 3.0 | 3.0 |
| Phenoxyethanol | 0.4 | 0.4 | 0.4 |
| Perfume | 0.35 | 0.35 | 0.35 |
| Dihydroxyacetone | 2.5 | 0.0 | 0.0 |
| Alloxane | 0.0 | 2.5 | 0.0 |
| Erythrulose | 0.0 | 0.0 | 2.5 |
| Adjuvant | 1.0 | 1.0 | 1.0 |
| Deionized water | to 100 | to 100 | to 100 |

*to pH of about 3.5

To assess the effect of the inventive adjuvants on coloration, the change in the Individual Typological Angle, with and without the adjuvant, was determined as a function of time using synthetic skin and in a manner similar to the one described in Example 1.

The change in the individual Typologic Angle (ITA°) for synthetic skin 24 hours after treatment with a variety of sunless tanning agents and adjuvants is shown in Table VI.

TABLE VI

| | | Δ ITA° for |
|---|---|---|
| | Δ ITA° for | amino methane |
| | taurine vs. | sulfonic acid |
| Sample | no adjuvant | vs. no adjuvant |
| E10 | 12.8 | 7.8 |
| E11 | 6.3 | 6.3 |
| E12 | 1.6 | 10.0 |

The results in Table VI indicate that the inventive adjuvants are effective in boosting the coloration within a ready to use composition.

### Example 5

The storage stability of DHA in the presence of the adjuvants of this invention was assessed at 35° C. for up to 20 days. During storage, remaining DHA percentage was determined via Ultra performance liquid chromatography using an apparatus made available by Acquity (UPLC). The assessed samples (i.e., ready-to-use compositions) are shown in Table VII.

TABLE VII

| Sample | | | | | |
|---|---|---|---|---|---|
| Ingredient | C5 | C6 | E13 | E14 | E15 |
| DHA | 2.5 | 2.5 | 2.5 | 2.5 | 2.5 |
| Glycine | 0 | 2.5 | 0 | 0 | 0 |
| AMSA | 0 | 0 | 2.5 | 0 | 0 |
| Taurine | 0 | 0 | 0 | 2.5 | 0 |
| Homotaurine | 0 | 0 | 0 | 0 | 2.5 |
| *Dove ® | balance | balance | balance | balance | balance |
| Energy Glow | | | | | |

*commercially available

Chemical stability of DHA as gauged by remaining percentage of DHA upon storage is reported in Table VIII. The lower amount of DHA means the faster consumption of DHA and lower stability.

TABLE VIII

| | Weight Percent DHA |
|---|---|
| Sample | Remaining After 20 Days |
| C5 | 2.25 |
| C6 | 1.25 |
| E13 | 1.60 |
| E14 | 2.20 |
| E15 | 2.20 |

The results unexpectedly demonstrate that ready-to-use compositions made with adjuvant consistent with this invention are more stable since more active, like DHA, remains in the composition after storage.

What is claimed is:

1. A composition comprising:
(a) a sunless tanning agent; and
(b) an adjuvant for the sunless tanning agent, the adjuvant comprising a nucleophile functionalized sulfonic acid.

2. The composition according to claim 1 wherein the sunless tanning agent is dihydroxyacetone, melanin, mehakanni, (eclipta alba), methyl glyoxal, erythrulose, alloxan, 2,3-dihydroxysuccindialdehyde or a mixture thereof.

3. The composition according to claim 1 wherein the adjuvant for the sunless tanning agent is represented by the formula:
Nu-X—SO₃H
further wherein:
X is a C₁₋₁₀ alkyl or alkenyl bridging radical;
Nu is SR, CN, I, OR, Cl or —NHR
and each R is independently H or a C₁₋₁₀ substituted or unsubstituted alkyl.

4. The composition according to claim 1 wherein the adjuvant for the sunless tanning agent is represented by the formula: 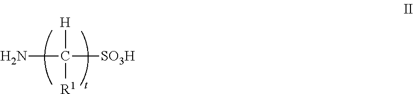
wherein R¹ is H or a C₁₋₁₀ substituted or unsubstituted alkyl group, and t is an integer from about 1 to about 8.

5. The composition according to claim 1 wherein the adjuvant for the sunless tanning agent is aminomethanesulfonic acid, 2-aminoethanesulfonic acid, 3-propanesulfonic acid or a mixture thereof.

6. The composition according to claim 1 wherein the sunless tanning agent is dihydroxyacetone which makes up from about 0.025 to about 35% by weight of the composition.

7. The composition according to claim 1 wherein the adjuvant for the sunless tanning agent is 2-aminoethanesulfonic acid which makes up from about 0.025 to about 35% by weight of the composition.

8. The composition according to claim 1 wherein the composition is an emulsion.

9. The composition according to claim 1 wherein the composition is an end use composition for a consumer which further comprises emulsifier, preservative, water, fragrance, vitamin, colorant, sunscreen or a mixture thereof.

10. The composition according to claim 1 wherein the composition comprises less than about 5% by weight glycine.

11. The composition according to claim 1 wherein the composition comprises no glycine.

12. The composition according to claim 1 wherein the composition is packaged in a dual compartment package.

13. The composition according to claim 1 wherein the composition is packaged in a single compartment package.

14. A method for imparting a sunless tan to skin comprising the steps of:
a) applying to skin the composition of claim 1; and
b) distributing the composition around the skin.
